(19) United States
(12) Patent Application Publication
(10) Pub. No.: US 2015/0123976 A1
(43) Pub. Date: May 7, 2015

(54) METHODS AND APPARATUS FOR INTERACTIVE DISPLAY OF IMAGES AND MEASUREMENTS

(71) Applicant: Charles River Laboratories, Inc., Wilmington, MA (US)

(72) Inventors: Lucas C. Siler, Shoreline, WA (US); Robert A. LoBosco, Bellevue, WA (US); Richard E. Long, Lee's Summit, MO (US); Yanning Zhu, Snoqualmie, WA (US); Courosh Mehanian, Redmond, WA (US)

(21) Appl. No.: 14/324,589

(22) Filed: Jul. 7, 2014

Related U.S. Application Data
(63) Continuation of application No. 13/025,768, filed on Feb. 11, 2011, now abandoned.
(60) Provisional application No. 61/366,067, filed on Jul. 20, 2010.

(51) Int. Cl. G06T 11/20; A61B 5/00

(52) U.S. Cl. CPC G06T 11/206; A61B 5/4845; G06T 2210/41
USPC 345/440

(57) ABSTRACT

Methods and apparatus for the interactive display of images and measurements of samples. A user may interact with embodiments of the present invention to display measurements for a set of biological samples and subsequently display images associated with particular biological samples of interest. For example, selecting data indicators in the set may result in the display of an image associated with a biological sample as well as one or more overlays presenting information of interest as well as controls affecting the display of the image or the information.

### CROSS-REFERENCE TO RELATED APPLICATIONS

This application claims priority to U.S. provisional patent appl. No. 61/366,067, filed on Jul. 20, 2010, which is incorporated herein by reference as if set forth in its entirety.

### FIELD OF THE INVENTION

The present invention relates to methods and apparatus for interactive displays, and in particular interactive user interfaces for the presentation of images and measurements to users of an electronic device.

### BACKGROUND OF THE INVENTION

Toxicologic pathology is the study of functional and structural changes induced in cells, tissues and organs by external stimuli such as drugs and toxins. Toxicologic studies are helpful to assessing the safety of drugs, vaccines, and other chemicals. A typical toxicologic study involves the controlled administration of at least one substance to a population of test animals. Tissue is harvested from the population using surgical processes such as necropsy. The harvested tissue is typically stained to improve the visibility of various tissue components. After processing, the tissue is mounted on a transparent substrate for viewing or digital imaging. By viewing the specimens, a diagnostician can identify the effects of the administered substance on the members of the test population.

The diagnostician faces several challenges as he or she studies specimen images. Different laboratories may process samples using different processes that may result in variations in color, contrast, or hue. The same variations may even arise in tissues processed in the same laboratory, for example, between tissues processed by different technicians or under different conditions. The diagnostician must exercise his or her judgment to distinguish between artifacts and clinically-significant features. When the diagnostician is reviewing a set of hundreds or even thousands of samples, human fallibility may cause artifacts to be deemed clinically significant features and vice versa.

Accordingly, there is a need for methods and apparatus for interactive display of images and measurements that help diagnosticians study and understand the biological samples.

### SUMMARY OF THE INVENTION

Embodiments of the present invention provide methods and apparatus for the interactive display of images and measurements of samples, such as biological samples. A user may interact with embodiments of the present invention to display statistics for a set of biological samples and subsequently display images associated with particular biological samples of interest. For example, selecting data indicators in the set may result in the display of an image associated with a biological sample as well as one or more overlays presenting information of interest as well as controls affecting the display of the image or the information.

One application for embodiments of the present invention is an interface for reviewing pathology-related measurements that are automatically extracted from images of tissue specimens for drug-safety toxicologic pathology studies. More generally, embodiments of the present invention can be used as an interface for reviewing any set of measurements from a plurality of source locales. The measurements may be human-generated or automatically-generated, and usually admit of a baseline or expected range of values.

In one aspect, embodiments of the present invention provide a computing device having a processing unit executing computer-readable instructions stored in the memory of the computing device for performing a method of presenting an integrated user interface on a display. The method may include presenting a two-dimensional arrangement of indicators on the display, with each indicator being representative of a sample of a variable type taken from a source and the appearance of each indicator varying based on a comparison of at least one measurement of the sample against a baseline value. Indicators representing samples from the same source may be arranged along a first axis and indicators representing samples of the same variable type are arranged along a second axis. An expandable indicator of at least one variable type may be registered along the second axis, and upon receipt of a selection of the expandable indicator, expanded indicators relating to at least one measurement for each source relating to that variable type may be displayed.

In various embodiments, the sample is a biological sample and the variable type is a tissue type. In other embodiments, the baseline values are based on domain knowledge or a historical normal group. The indicators may have a first appearance to identify samples within a historical normal range of the at least one measurement and a second appearance to identify samples lying outside the historical normal range of the at least one measurement.

In another embodiment, the method further includes selecting at least some of the expanded indicators to display a related measurement of the source of the sample relevant to the selected variable type. In another embodiment, the method further comprises receiving a selection of a measurement associated with a selected variable type, and registering a plurality of indicators on a two-dimensional plane, where each indicator represents the selected measurement for each sample. This presentation format is traditionally called a scatter plot. In various embodiments, the displayed indicators are presented distributed substantially evenly along the first axis in order of increasing or decreasing value and according to value along the second axis. The displayed indicators may be presented in one or more series representing different groupings of the samples, and each series may be distributed across a substantially contiguous portion of the first axis or across separate, contiguous, non-overlapping portions of the first axis. The method may further include receiving a selection of a displayed indicator representing a measurement and displaying an image of the sample associated with the displayed indicator.

In another aspect, embodiments of the present invention provide a computing device having a processing unit executing computer-readable instructions stored in the memory of the computing device for performing a method of presenting an integrated user interface on a display. The method may include registering a plurality of indicators on a two-dimensional plane, where each indicator represents at least one measurement for a sample of a variable type taken from a source and the registered indicators are presented distributed along a first axis in order of increasing or decreasing value and according to value along a second axis. A first area of the two-dimensional plane may have a first appearance and contain indicators associated with samples having measurement values within a specified interval around the baseline value.

In various embodiments, the sample is a biological sample and the variable type is a tissue type. In one embodiment, the specified interval is one standard deviation of the baseline value. The baseline value may be based on a historical normal group, which may include historical values removing vehicle effects and historical values removing protocol effects. In another embodiment, a second area of the two-dimensional plane has a second appearance that is different from the first appearance, the second area containing indicators associated with samples having measurement values outside a specified interval around the baseline value. The interval may be one standard deviation of the baseline value, such as a historical normal value. In another embodiment, the registered indicators are presented distributed substantially evenly along the first axis.

In still another aspect, embodiments of the present invention provide a computing device comprising a processing unit executing computer-readable instructions stored in the memory of the computing device for performing a method of presenting an integrated user interface on a display. The method may include displaying at least a portion of an image associated with a sample and displaying statistical information concerning at least one quantitative and spatially varying property of the sample as an overlay to the displayed portion of the image.

In other embodiments, the statistical information is summarized and displayed as an ordered graph. In another embodiment, the statistical information is summarized and displayed as a histogram. In another embodiment, different portions of the image are displayed in response to a user input. In still another embodiment, the method further includes displaying a selection menu in the foreground of the display. The selection menu may itself be semi-transparent. The sample may be a biological sample. In yet another embodiment, the statistical information overlay is semi-transparent.

The foregoing and other features and advantages of the present invention will be made more apparent from the description, drawings, and claims that follow.

### BRIEF DESCRIPTION OF DRAWINGS

The advantages of the invention may be better understood by referring to the following drawings taken in conjunction with the accompanying description in which:

FIG. 1 is an illustration of a user interface presenting indicators for a set of biological samples in accord with an embodiment of the present invention;

FIG. 2 presents the interface of FIG. 1 when expandable indicators, each associated with a tissue type, have been selected;

FIG. 3 is a depiction of a two-dimensional scatter plot of measurements for a set of biological samples in accord with an embodiment of the present invention;

FIG. 4 is another depiction of a two-dimensional scatter plot of measurements for a set of biological samples in accord with the present invention;

FIG. 5 is a depiction of an image associated with a particular biological sample having a frame displaying the entire image of the biological sample;

FIG. 6 illustrates the presentation of a plurality of images, each associated with a particular biological sample;

FIG. 7 is a depiction of an image associated with a particular biological sample with an overlay presenting information of interest concerning the sample in the form of a spatially varying map;

FIG. 8 is a depiction of an image associated with a particular biological sample having an overlay presenting information of interest concerning the sample in the form of an ordered graph;

FIG. 9 is a depiction of an image associated with a particular biological sample having an overlay presenting information of interest concerning the sample and an overlay controlling the display of the image and the information; and

FIG. 10 is a flowchart of an embodiment of a method for presenting an integrated user interface on a display in accord with the present invention.

In the drawings, like reference characters generally refer to corresponding parts throughout the different views. The drawings are not necessarily to scale, emphasis instead being placed on the principles and concepts of the invention.

### DETAILED DESCRIPTION OF THE INVENTION

Embodiments of the present invention provide a general interface for reviewing large numbers of measurements that have been extracted from a plurality of sources. The measurements may be human-generated or automatically-generated, and usually admit of a baseline or expected range of values. The sources may consist of tissue specimens or images thereof, manufactured items or images thereof, geographical locations or images thereof, other variable types, etc. The following discussion uses a toxicologic pathology study as an example for clarity, but it is to be understood that this example does not limit the domain of applicability of the current invention in any manner.

A toxicologic pathology study involves the administration of a drug to a plurality of animals, usually in various dose groups, including a control group. After the animals are sacrificed, typically one or more tissues are sectioned, stained, and mounted on microscope slides. The slides or digital images thereof may then be either reviewed by pathologists or by an automated pathology system, or a combination thereof.

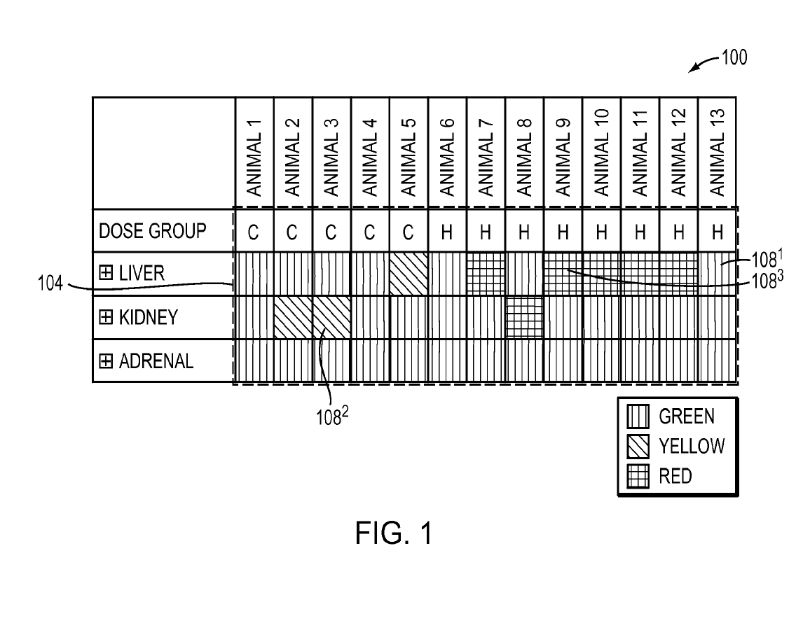

With reference to FIG. 1, user interface 100 presents measurement information concerning a set of biological samples obtained from a toxicologic pathology study. The interface 100 may include a two-dimensional array (or grid/plane) 104 of disposition indicators 108. The term “disposition” refers to the comparison of the measurements against the baseline. Each disposition indicator 108 may represent at least one measurement of a biological sample of a tissue type taken from a source.

In this embodiment, the disposition indicators 108 are organized so that the disposition indicators 108 originating from the same biological source (e.g., “Animal 1”) are presented in the same column (or same location on a first axis). The disposition indicators 108 representing biological samples of the same tissue type (e.g. Liver) may be presented in the same row (or same location on a second axis).

Continuing with the example of a toxicologic pathology study, FIG. 1 shows a high-level summary of a hypothetical study in which microscope slides of the liver, kidney, and adrenal sections of 13 animals have been obtained. The first five animals are in the control group, and the remaining eight animals have received a high dose of the drug administered by way of a vehicle, e.g., usually by injection or orally. The animals in the control group were not given any drug, but received the same administration vehicle.

The appearance of each disposition indicator 108 varies based on a comparison of the at least one measurement of the biological sample against a baseline value. The baseline value may be selected, for example, using domain knowledge or based on historical data values, such as a historical normal group. In one embodiment, a green disposition indicator 108¹ can be used to indicate that the measurement is within a specified interval around the baseline value, such as one standard deviation of the baseline. A yellow disposition indicator 108² may be used to indicate that a measurement for a sample in the control group exceeds the specified range, e.g., being more than one standard deviation away from the baseline value. A red disposition indicator 108³ may similarly be used to indicate that a measurement for a sample in the test group exceeds, e.g., one standard deviation from the baseline value. The colors in the array 104 may be succinctly summarized: green means normal disposition; yellow means non-normal disposition for the control group; and red means non-normal disposition for the dosed group.

Continuing this example, each red, yellow, or green disposition indicator 108 in FIG. 1 represents one or more tissue sections per animal. In many cases, there is only one tissue per animal, for example, one liver lobe section, one kidney section, one adrenal section, or a heart section, etc. In some cases, however, multiple tissues are extracted, sectioned, and mounted for the same animal, for example, two liver lobe sections, the left and right kidney sections, or the left and right adrenal sections. Each tissue section may be subjected to one or more types of measurements that have pathological significance. For example, liver images may be measured to obtain vacuolation density and hepatocyte nuclear density. As discussed above, a green cell 108¹ in the table means that none of the measurements for none of the tissue sections for that animal differed significantly from the baseline values. For the control group, the color yellow 108² indicates that at least one of the measurements for one of the sections differed significantly from the baseline. For the high-dose group, the color red 108³ indicates that at least one of the measurements for one of the sections differed significantly from the baseline.

In this high-level view, the salient information stands out clearly. For this example, the table shows at a glance that the adrenals of the control and high-dose groups were unaffected in the study. The liver of one animal in the control group was different than baseline, while five out of eight livers receiving a high-dose were significantly impacted. For the kidney variable, two animals in the control group were affected, while only one in the high-dose group was affected. The table conveys that this compound had a significant impact on the livers of the high-dose group, either compared to a baseline or to the control group; the adrenals were unaffected; and the effect on the kidneys is about the same for control and high-dose groups and thus consistent with a vehicle-related effect.

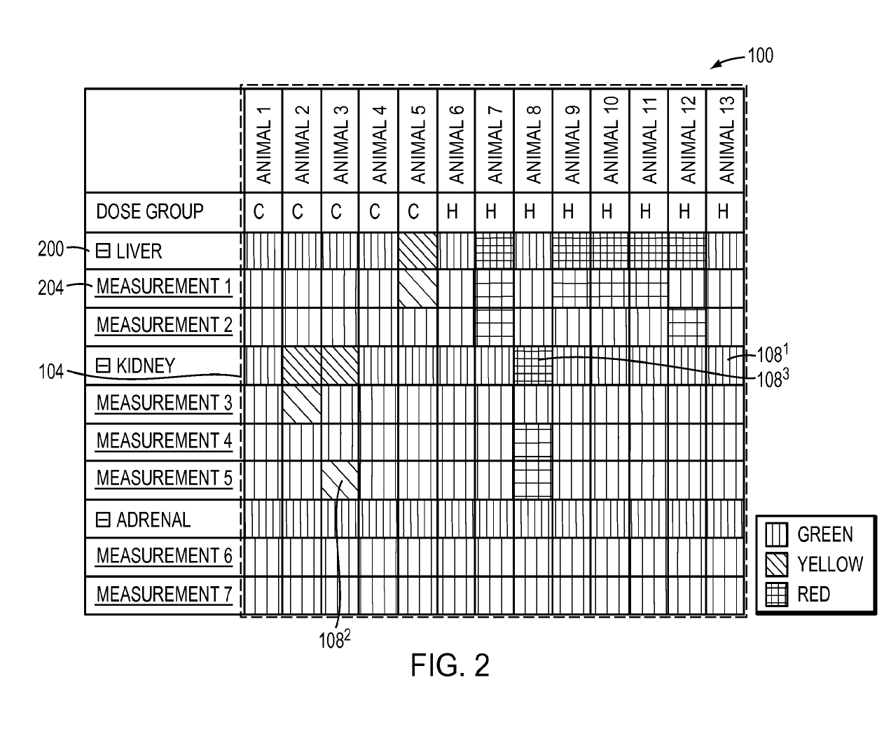

With reference to FIG. 2, some rows include an expandable indicator 200 of a tissue type. When an expandable indicator 200 is selected by a user, e.g., by pointing at the indicator 200 and providing a mouse click, the indicator 200 may expand to list, in a plurality of rows, at least one expanded indicator 204, which may be a measurement associated with the tissue type.

Continuing with the previous example, this first level of drill down analysis is to view the types of measurements that make up the combined disposition for each tissue. By expanding the expandable indicator 200, the user interface may show the names of each measurement for each tissue type (i.e., liver, kidney, and adrenal). In FIG. 2, the number of measurements for each tissue is arbitrary and the names of the measurements are generic for this hypothetical example. In other applications, the number and nature of the measurements may be determined by the application domain and the mechanism generating the measurements. As discussed above, when one of the individual measurements in a composite measurement differs from a baseline value, the composite measurement can be colored to indicate the deviation of the component measurement. For example, the kidney tissue of animal 8 is marked as different from baseline (i.e., red) because at least one of measurements 3, 4, and 5 is different from the baseline (i.e., red).

There are various methods of obtaining the baseline threshold against which the study measurements are compared. In one embodiment, the baseline threshold is based on domain knowledge. In another embodiment, the baseline threshold is computed from a set of samples (e.g., tissues, geographical locations, manufactured parts, other variables, etc.) that have been deemed “normal” by a domain expert. The normal set of samples is referred to as the historical normal group. The samples in the historical normal group may be selected to reflect values without vehicle and/or protocol effects. The historical normal group may be subjected to the same set of measurements as the study samples. A number of methods have been devised for computing the baseline threshold from the historical normal measurements, e.g., mean plus or minus two standard deviations, outlier analysis, etc. The particular method used will vary among embodiments; given the historical normal measurements, any reasonable method will suffice for determining a baseline threshold.

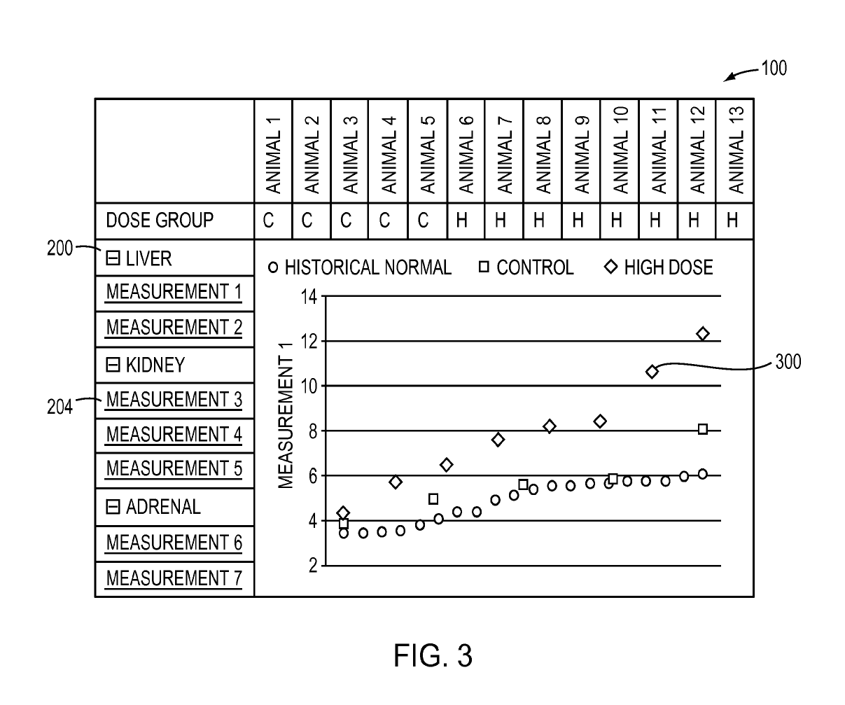

With reference again to FIG. 2, while interacting with the expandable indicators 200, a user may select an expanded indicator 204. In certain embodiments, the selection of an expanded indicator 204 results in display of measurement values associated with the expanded indicator 204 for each of the biological samples. One format suited to the display of such values is a two-dimensional scatter plot, with the measurements organized in ascending or descending numerical order, as is presented in FIG. 3.

With further reference to FIG. 3, the scatter plot allows for a quantitative analysis of the values for a particular measurement across the historical normal, control, and high-dose groups. In this embodiment, the samples are first sorted in order of increasing measurement value within each group, and then plotted as separate series with a common abscissa range, distributed across a substantially contiguous portion of the first axis. Continuing this example, it is obvious from the chart that the high-dose group is significantly different from either the control group or the historical normal group; this conclusion follows from the observation that the high-dose data measurement indicators lie significantly above the data measurement indicators for the other two groups.

Some embodiments of the present invention optionally provide a p-value for the two-sided significance test using the null hypothesis that the high-dose group is drawn from the same distribution as the historical normal group. Other embodiments provide a p-value for a two-sided significance test using the null hypothesis that the control group is not significantly different from the historical normal group.

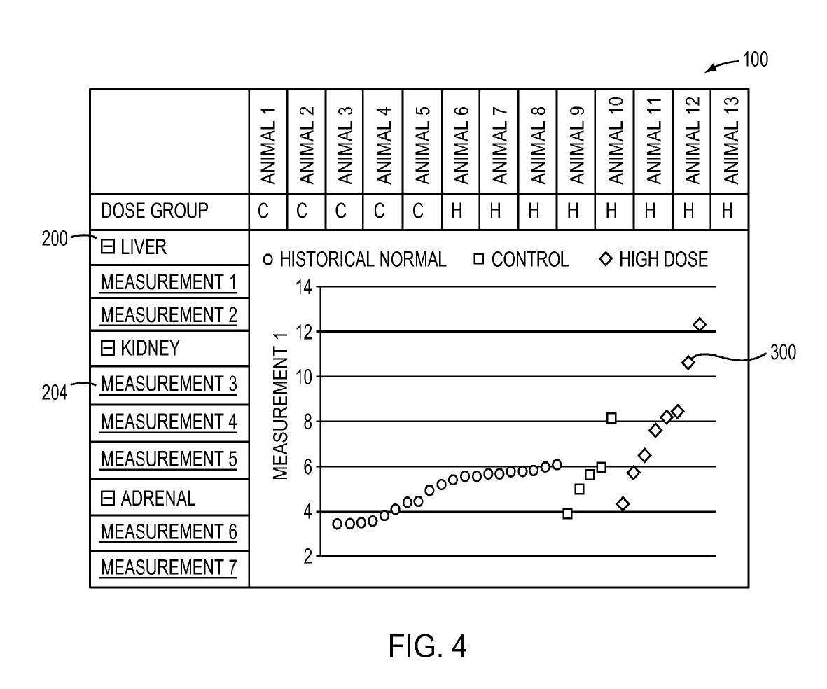

With reference to FIG. 4, the user may toggle the chart so that the series are not shown on top of each other as in FIG. 3, but instead are presented side-by-side, such that each series is distributed across separate, substantially contiguous, non-overlapping portions of the first axis. This version of the chart is complementary to the version shown in FIG. 3. Depending on the characteristics of the study measurements, the chart version shown in FIG. 4 may cause significant differences to be more readily apparent than the chart version shown in FIG. 3.

In another embodiment (not shown), the appearance of an area is modified (e.g., by shading) to indicate which measurement indicators on the scatter plot fall within a specified interval around a baseline value. For example, the measurement indicators in a series falling within one standard deviation of the baseline value may appear in a shaded rectangle on the scatter plot, while the measurement indicators outside one standard deviation of the baseline value may appear to be in an unshaded second area. In another embodiment, the specified interval may be based on a historical normal range as determined from the historical normal group. The historical normal group may include historical values removing vehicle effects and historical values removing protocol effects. The shaded and unshaded areas may also be reversed, and the measurements indicators may be presented substantially evenly along the first axis.

Each data measurement indicator 300 of the charts of FIG. 3 and FIG. 4 represents the result of aggregating a plurality of measurements from a microscope image of a tissue section. More generally speaking, every data measurement indicator 300 plotted on the chart is an aggregation of a plurality of measurements across its associated source locale. Various statistical methods may be chosen to aggregate the set of measurements into a single measurement, for example, mean, median, or other statistical aggregation formulas.

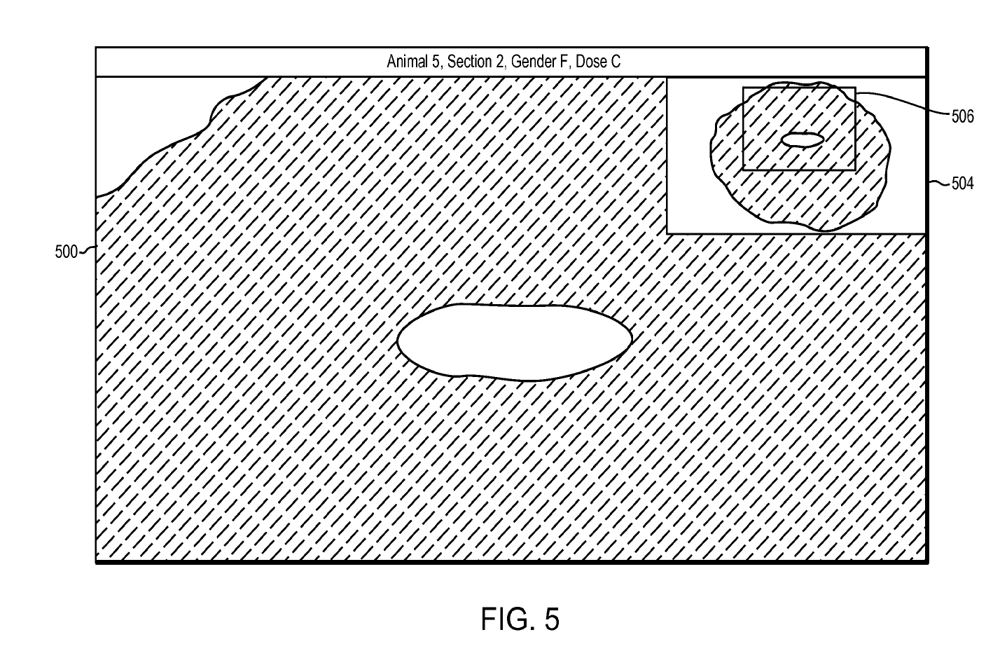

With reference to FIGS. 1-4, while interacting with the interface 100, a user may select an individual disposition indicator 108 or data measurement indicator 300. In certain embodiments, selection of a disposition indicator 108 or a data measurement indicator 300 results in the opening of a window 500 (either occupying part or the entirety of the display) as depicted in FIG. 5.

In one embodiment, the window 500 includes a frame 504 that displays a thumbnail view of the entire image associated with the window 500. In one embodiment, a rectangle 506 is used to indicate the portion of the associated image that is currently being displayed in the window 500.

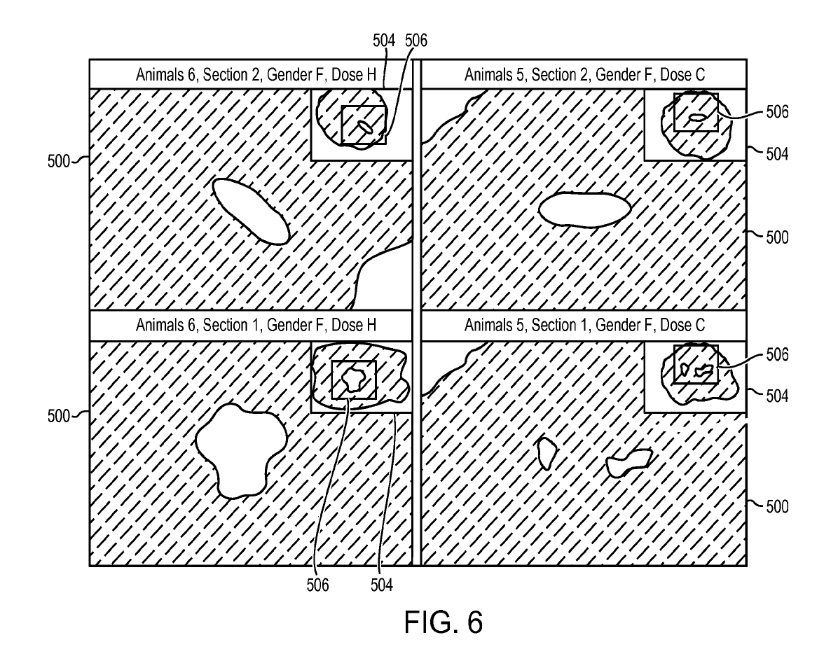

In another embodiment, a user may display multiple source images in windows 500 located side-by-side by activating more than one data measurement indicator 300, as is shown in FIG. 6. In a geographical locations example, this operation could correspond to viewing the locations superimposed on a map or satellite images of the terrain. Either of these could be shown whether measurements were made on the ground or directly extracted from satellite images. In the toxicologic pathology study example, this operation corresponds to viewing the digital microscope images of the animal tissues.

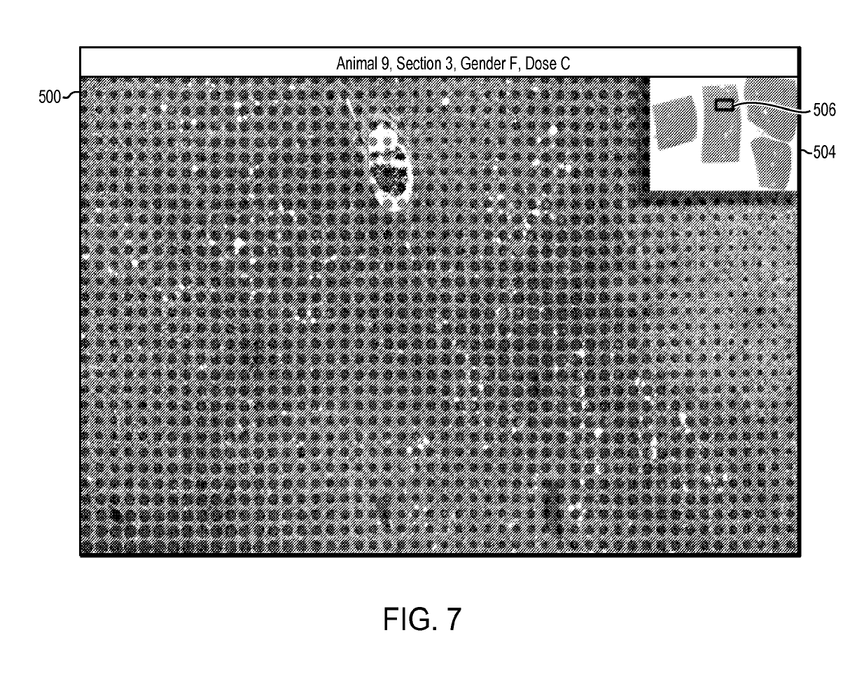

A further level of drill down analysis is to view the distribution of the measurements across a particular source locale. In a toxicologic pathology study, the user may select one of the data measurement indicators 300 on the graph because the aggregate measurement stands out from the baseline, or they may select one of the disposition indicators 108 because the disposition is non-normal, to see how the individual measurements that contributed to the aggregate measurement are distributed across the tissue. In particular, the user may be looking to identify “hotspots” in the tissue image. In one embodiment, the user activates a control that overlays a spatially varying, semi-transparent map on top of the source image as shown in FIG. 7. In other embodiments, the map may be opaque. As shown in FIG. 7, the overlaid map may be a regular grid of semi-transparent disks. The size of the overlaid disks at a particular location may indicate the local magnitude of the measurement according to a disk size lookup table. In another embodiment (not shown) the color of different areas of the overlaid map indicate the local magnitude of the measurement according to a color lookup table. In either embodiment, the user can see which sub-regions of the tissue have elevated levels of the measurement, and they can zoom in on those areas of interest for further analysis.

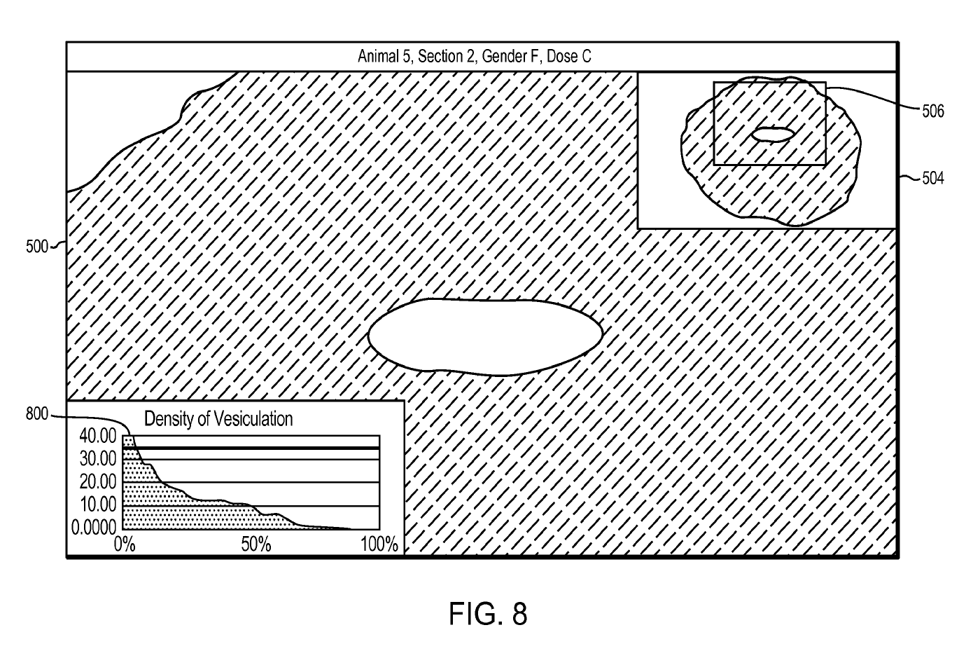

In addition to the spatial variation of the measurement across the source locale, the user may be interested to see the distribution of measurement values directly. In one embodiment, the user activates a control that displays a histogram of the measurement values across the tissue section image. In another embodiment, the user activates a control that displays a mini-plot 800 of the measurement values across the tissue section image in order of decreasing magnitude, as depicted as an inset on the tissue section image 500 in FIG. 8.

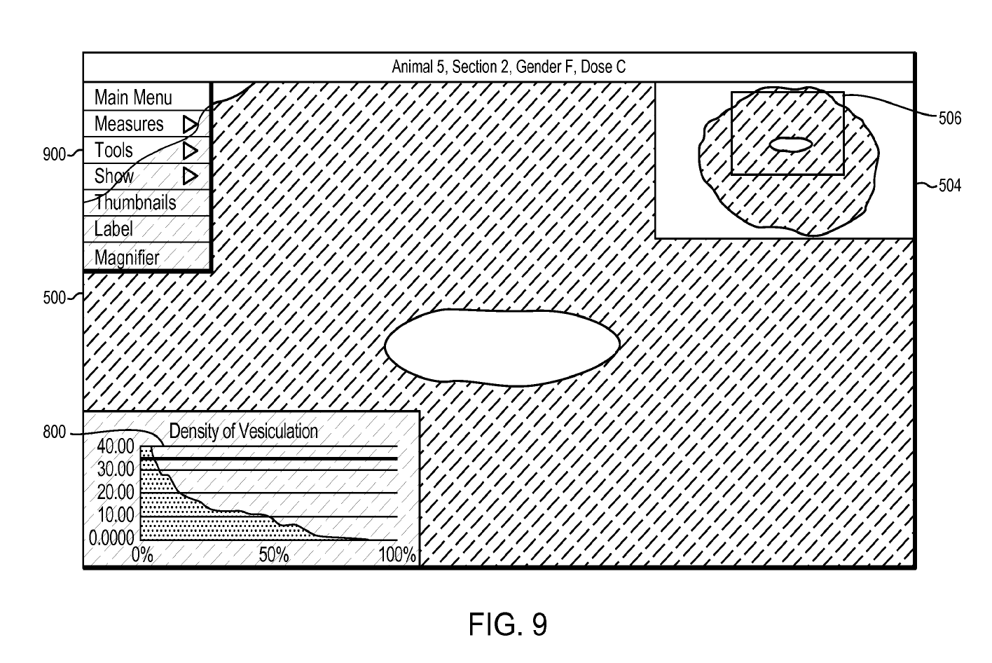

With reference to FIG. 9, in another embodiment of the present invention, the window 500 is displayed with a semi-transparent control overlay (or selection menu) 900 that determines how the image is displayed in the window 500 or what information is displayed in the frame 800. In other embodiments, the control overlay 900 may be opaque.

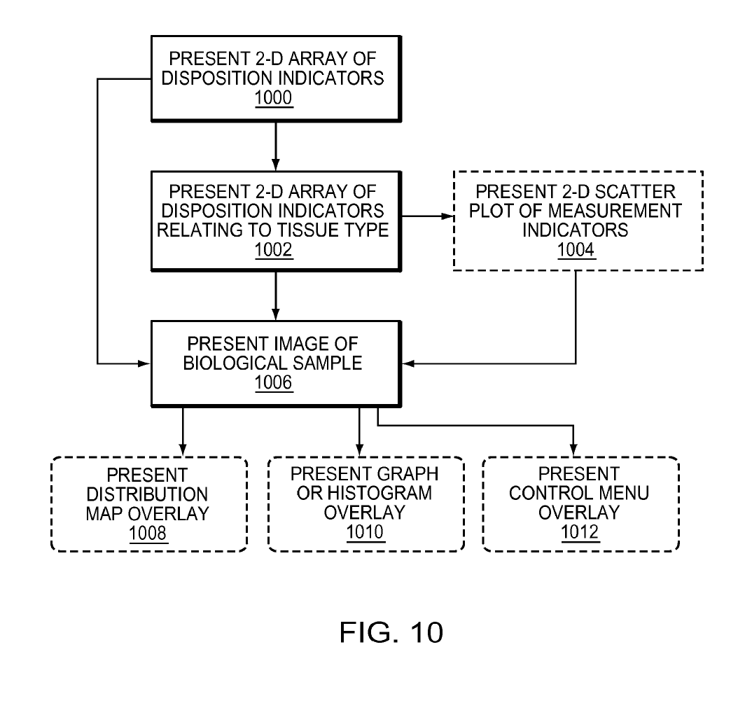

FIG. 10 is a flowchart presenting an embodiment of a method for the interactive display of images of biological samples according to the present invention. As discussed above, a two-dimensional array (or grid/plane) of indicators can be presented, with each indicator corresponding to the disposition of at least one measurement for a biological sample (Step 1000). This two-dimensional arrangement optionally may be expanded to present disposition indicators relating to tissue type (Step 1002). When an indicator is selected from either the original (from Step 1000) or the expanded (from Step 1002) two-dimensional arrangement of indicators, the system displays an image of the biological sample associated with the indicator (Step 1006). When a measurement indicator in the expanded two-dimensional arrangement (from Step 1002) is selected, the system presents a two-dimensional scatter plot of the measurements associated with the samples (Step 1004). When a measurement indicator in the two-dimensional scatter plot is selected, the system displays an image of the biological sample associated with the measurement indicator (Step 1006). The image of the biological sample optionally may be accompanied by an overlay containing information concerning the displayed image. The overlay may be in the form of a map (Step 1008), or in the form of an ordered graph or histogram (Step 1010). The map may be semi-transparent. Multiple overlays may be displayed at the same time. Multiple ordered graphs or histograms corresponding to different measurements on the same sample may be displayed at the same time. If the image of the biological sample is displayed by way of the two-dimensional array of indicators (corresponding to going directly from Step 1000 to Step 1006 in FIG. 10), an intermediate step of selecting the particular measurement may be utilized before overlays are optionally displayed. On the other hand, if the image of the biological sample is displayed by way of the two-dimensional scatter plot (corresponding to the path from Step 1004 to Step 1006), the overlay may relate to the measurements in the scatter plot. The particular measurement and statistical information in the overlays may be selected via an overlaid control menu (Step 1012).

The interactive user interfaces described herein may be implemented, in various embodiments, in a computing device. The device may be any type of device capable of receiving, rendering and displaying graphical user interfaces, such as, for example, a personal computer (PC), a laptop computer, a workstation, a smartphone, etc. The interactive user interfaces may be displayed as the device executes software code using any type of computer instruction type suitable, such as, for example, Java, C, C++, Visual Basic, etc., using, for example, conventional or object-oriented techniques. The software code may be stored as a series of instructions or commands on a computer readable medium, such as a random access memory (RAM), a read only memory (ROM), a solid-state drive (flash RAM), a magnetic medium such as a hard drive or a floppy disk, or an optical medium such as a CD-ROM.

It will therefore be seen that the foregoing represents an advantageous approach to the interactive display of images of biological samples and their corresponding measurements. The terms and expressions employed herein are used as terms of description and not of limitation and there is no intention, in the use of such terms and expressions, of excluding any equivalents of the features shown and described or portions thereof, and it is recognized that various modifications are possible within the scope of the invention claimed.

1. A computing device comprising a processing unit executing computer-readable instructions stored in memory of the computing device for performing a method of presenting an integrated user interface on a display, the method comprising:
presenting a two-dimensional arrangement of indicators on the display, wherein each indicator is representative of a sample of a variable type taken from a source, and the appearance of each indicator varies based on a comparison of at least one measurement of the sample against a baseline value, wherein indicators representing samples from the same source are arranged along a first axis and indicators representing samples of the same variable type are arranged along a second axis; and
registering an expandable indicator of at least one variable type along the second axis, wherein upon receipt of a selection of the expandable indicator, expanded indicators relating to the at least one measurement for each source relating to that variable type are displayed.

2. The computing device of claim 1, wherein the sample comprises a biological sample.

3. The computing device of claim 1, wherein the variable type comprises a tissue type.

4. The computing device of claim 1, wherein the baseline value is based on domain knowledge.

5. The computing device of claim 1, wherein the baseline value is based on a historical normal group.

6. The computing device of claim 1, wherein the indicators have a first appearance to identify samples within a historical normal range of the at least one measurement and a second appearance to identify samples lying outside the historical normal range of the at least one measurement.

7. The computing device of claim 1, wherein the method further comprises selecting at least some of the expanded indicators to display a related measurement of the source of the sample relevant to the selected variable type.

8. The computing device of claim 1, wherein the method further comprises receiving a selection of a measurement associated with a selected variable type, and registering a plurality of indicators on a two-dimensional plane, where each indicator represents the selected measurement for each sample and the displayed indicators are presented distributed substantially evenly along the first axis in order of decreasing or increasing value and according to value along the second axis.

9. The computing device of claim 8, wherein the displayed indicators are presented in one or more series representing different groupings of the samples, and wherein each series is distributed across a substantially contiguous portion of the first axis.

10. The computing device of claim 8, wherein the displayed indicators are presented in one or more series representing different groupings of the samples, and wherein each series is distributed across separate, contiguous, non-overlapping portions of the first axis.

11. The computing device of claim 8, wherein the method further comprises receiving a selection of a displayed indicator representing a measurement and displaying an image of the sample associated with the displayed indicator.

12. A computing device comprising a processing unit executing computer-readable instructions stored in memory of the computing device for performing a method of presenting an integrated user interface on a display, the method comprising:
registering a plurality of indicators on a two-dimensional plane, where each indicator represents at least one measurement for a sample of a variable type taken from a source and the registered indicators are presented distributed along a first axis in order of decreasing or increasing value and according to value along a second axis; and
wherein a first area of the two-dimensional plane has a first appearance, the first area containing indicators associated with samples having measurement values within a specified interval around the baseline value.

13. The computing device of claim 12, wherein the sample comprises a biological sample.

14. The computing device of claim 12, wherein the variable type comprises a tissue type.

15. The computing device of claim 12, wherein the specified interval is one standard deviation of the baseline value.

16. The computing device of claim 12, wherein the baseline value is based on a historical normal group.

17. The computing device of claim 16, wherein the historical normal group consists of historical values removing vehicle effects and historical values removing protocol effects.

18. The computing device of claim 12, wherein a second area of the two-dimensional plane has a second appearance that is different from the first appearance, the second area containing indicators associated with samples having measurement values outside a specified interval around the baseline value.

19. The computing device of claim 12, wherein the registered indicators are presented distributed substantially evenly along the first axis.

20. A computing device comprising a processing unit executing computer-readable instructions stored in memory of the computing device for performing a method of presenting an integrated user interface on a display, the method comprising:
displaying at least a portion of an image associated with a sample; and
displaying statistical information concerning at least one quantitative and spatially varying property of the sample as an overlay to the displayed portion of the image.

21.-25. (canceled)
